(19) United States
(12) Patent Application Publication
(10) Pub. No.: US 2016/0207904 A1
(43) Pub. Date: Jul. 21, 2016

(54) CRYSTALLINE 2-HYDROXY-6-((2-(1-ISOPROPYL-1H-PYRAZOL-5-YL)PYRIDIN-3-YL)METHOXY)BENZALDEHYDE ANSOLVATE SALTS

(71) Applicant: GLOBAL BLOOD THERAPEUTICS, INC., South San Francisco, CA (US)

(72) Inventor: Zhe LI, San Francisco, CA (US)

(21) Appl. No.: 14/914,597

(22) PCT Filed: Aug. 25, 2014
(86) PCT No.: PCT/US14/52576

§ 371 Date: Feb. 25, 2016

Related U.S. Application Data
(60) Provisional application No. 61/962,309, filed on Aug. 27, 2013.

(51) Int. Cl. C07D 401/04

(52) U.S. Cl. CPC C07D 401/04

(57) ABSTRACT

Disclosed are crystalline ansolvate salts of 2-hydroxy-6-((2-(1-isopropyl-1H-pyrazol-5-yl)pyridin-3-yl)methoxy)benzaldehyde (or Compound 1), such as the hydrochloride Form I.

### CROSS-REFERENCE TO RELATED APPLICATIONS

This application claims the benefit under 35 U.S.C. §119(e) to U.S. provisional application Ser. No. 61/962,309, which was converted from U.S. nonprovisional application Ser. No. 14/011,601, filed Aug. 27, 2013, both of which are incorporated herein by reference in their entirety.

### BACKGROUND

2-Hydroxy-6-((2-(1-isopropyl-1H-pyrazol-5-yl)pyridin-3-yl)methoxy)benzaldehyde is a compound having the formula:

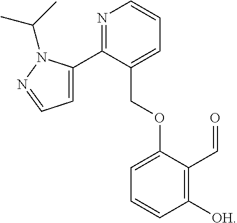 [SMILES: CC(C)n1nccc1-c1ncccc1COc1cccc(O)c1C=O]

Sickle cell disease is a disorder of the red blood cells, found particularly among those of African and Mediterranean descent. The basis for sickle cell disease is found in sickle hemoglobin (HbS), which contains a point mutation relative to the prevalent peptide sequence of hemoglobin (Hb).

Hemoglobin (Hb) transports oxygen molecules from the lungs to various tissues and organs throughout the body. Hemoglobin binds and releases oxygen through conformational changes. Sickle hemoglobin (HbS) contains a point mutation where glutamic acid is replaced with valine, allowing HbS to become susceptible to polymerization to give the HbS containing red blood cells their characteristic sickle shape. The sickled cells are also more rigid than normal red blood cells, and their lack of flexibility can lead to blockage of blood vessels. A need exists for therapeutics that can treat disorders that are mediated by Hb or by abnormal Hb such as HbS, such as 2-hydroxy-6-((2-(1-isopropyl-1H-pyrazol-5-yl)pyridin-3-yl)methoxy)benzaldehyde hydrochloride.

When used for treating humans, it is important that a crystalline form of a therapeutic agent, like 2-hydroxy-6-((2-(1-isopropyl-1H-pyrazol-5-yl)pyridin-3-yl)methoxy)benzaldehyde, or a salt thereof, retains its polymorphic and chemical stability, solubility, and other physicochemical properties over time and among various manufactured batches of the agent. If the physicochemical properties vary with time and among batches, the administration of a therapeutically effective dose becomes problematic and may lead to toxic side effects or to ineffective therapy, particularly if a given polymorph decomposes prior to use, to a less active, inactive, or toxic compound. Therefore, it is important to choose a form of the crystalline agent that is stable, is manufactured reproducibly, and has physicochemical properties favorable for its use as a therapeutic agent.

However, the art remains unable to predict which crystalline form of an agent will have a combination of the desired properties and will be suitable for human administration, and how to make the agent in such a crystalline form.

### SUMMARY

It has now been discovered that 2-hydroxy-6-((2-(1-isopropyl-1H-pyrazol-5-yl)pyridin-3-yl)methoxy)benzaldehyde (or Compound 1) salts and, in particular, the hydrochloride salt, can be obtained as a crystalline ansolvate, referred to here as crystalline Form I. Surprisingly, the hydrochloride ansolvate demonstrates superior stability and other physicochemical properties compared to the solvate crystalline hydrochloride Forms II and III, as disclosed herein.

Accordingly, in one aspect, this invention provides for crystalline Compound 1 ansolvate salts and, in particular, the hydrochloride salt (crystalline Form I). In one embodiment, the crystalline Compound 1 ansolvate salt does not undergo polymorphic transformation under conditions suitable for manufacturing and storing the crystalline ansolvate forms. In another embodiment, the crystalline Compound 1 hydrochloride ansolvate is characterized by an endothermic peak at (196±2) C as measured by differential scanning calorimetry. In another embodiment, the crystalline Compound 1 hydrochloride ansolvate is characterized by the substantial absence of thermal events at temperatures below the endothermic peak at (196±2) ° C. as measured by differential scanning calorimetry. In another embodiment, the crystalline Compound 1 hydrochloride ansolvate is characterized by an X-ray powder diffraction peak (Cu Kα radiation at one or more of 11.6°, 14.6°, 15.8° or 19.0° 2θ. In another embodiment, the crystalline Compound 1 hydrochloride ansolvate is characterized by an X-ray powder diffraction pattern (Cu Ka radiation) substantially similar to that of FIG. 1A.

In another embodiment, the crystalline Compound 1 hydrochloride ansolvate is characterized by at least one X-ray powder diffraction peak (Cu Kα radiation) selected from 11.6°, 14.6°, 15.8° and 19.0° 2θ (each ±0.2° 2θ). In another embodiment, the crystalline Compound 1 hydrochloride ansolvate is characterized by at least two X-ray powder diffraction peaks (Cu Kα radiation) selected from 11.6°, 14.6°, 15.8° and 19.0° 2θ (each ±0.2° 2θ). In another embodiment, the crystalline Compound 1 hydrochloride ansolvate is characterized by at least three X-ray powder diffraction peaks (Cu Kα radiation) selected from 11.6°, 14.6°, 15.8° and 19.0° 2θ (each ±0.2° 2θ).

In another of its composition embodiments, this invention provides for a pharmaceutical composition comprising a pharmaceutically acceptable excipient and a crystalline Compound 1 ansolvate salt, such as the hydrochloride Form I.

In one of its method embodiments, this invention provides a method of preparing the solid crystalline Compound 1 ansolvate salt, such as the hydrochloride Form I

In yet another of its method embodiments, there are provided methods for increasing oxygen affinity of hemoglobin S in a subject, the method comprising administering to a subject in need thereof a therapeutically effective amount of a crystalline ansolvate salt of Compound 1, such as the hydrochloride Form I.

In yet another of its method embodiments, there are provided methods for treating oxygen deficiency associated with sickle cell anemia in a subject, the method comprising administering to a subject in need thereof a therapeutically effective amount of a crystalline ansolvate salt of Compound 1, such as the hydrochloride Form I

In all of such treatments, the dosing of crystalline ansolvate salt of Compound 1, such as the hydrochloride Form I, to the treated patient is already disclosed in the art.

### BRIEF DESCRIPTION OF THE DRAWINGS

FIG. 1A is a XRPD profile of crystalline Compound 1 hydrochloride ansolvate

Form I.

FIG. 1B is a XRPD profile of crystalline Compound 1 hydrochloride Form II.

FIG. 2 is a TGA profile of crystalline Compound 1 ansolvate Form I.

FIG. 3 is a DSC profile of crystalline Compound 1 ansolvate Form I.

FIG. 4 is a DVS profile of crystalline Compound 1 Form I.

FIG. 5 is a TGA profile of Form II.

FIG. 6 is a DSC profile of Form II.

### DETAILED DESCRIPTION

As noted above, this invention is directed, in part, to a stable crystalline ansolvate of Compound 1 salts and, in particular, the hydrochloride salt. However, prior to discussing this invention in further detail, the following terms will be defined.

### Definitions

As used herein, the following terms have the following meanings

The singular forms “a,” “an,” and “the” and the like include plural referents unless the context clearly dictates otherwise. Thus, for example, reference to “a compound” includes both a single compound and a plurality of different compounds.

The term “about” when used before a numerical designation, e.g., temperature, time, amount, and concentration, including a range, indicates approximations which may vary by ±10%, ±5% or ±1%.

“Administration” refers to introducing an agent into a patient. A therapeutic amount can be administered, which can be determined by the treating physician or the like. An oral route of administration is preferred. The related terms and phrases administering” and “administration of”, when used in connection with a compound or pharmaceutical composition (and grammatical equivalents) refer both to direct administration, which may be administration to a patient by a medical professional or by self-administration by the patient, and/or to indirect administration, which may be the act of prescribing a drug. For example, a physician who instructs a patient to self-administer a drug and/or provides a patient with a prescription for a drug is administering the drug to the patient. In any event, administration entails delivery to the patient of the drug.

The “crystalline ansolvate” of Compound 1 hydrochloride is a crystalline solid form of 2-hydroxy-6-((2-(1-isopropyl-1H-pyrazol-5-yl)pyridin-3-yl)methoxy)benzaldehyde hydrochloride, such as, e.g., the crystalline Form I. The Form I crystal lattice is substantially free of solvents of crystallization. However, any solvent present is not included in the crystal lattice and is randomly distributed outside the crystal lattice. Therefore, Form I crystals in bulk may contain, outside the crystal lattice, small amounts of one or more solvents, such as the solvents used in its synthesis or crystallization. As used above, “substantially free of” and “small amounts,” refers to the presence of solvents preferably less that 10,000 parts per million (ppm), or more preferably, less than 500 ppm.

“Characterization” refers to obtaining data which may be used to identify a solid form of a compound, for example, to identify whether the solid form is amorphous or crystalline and whether it is unsolvated or solvated. The process by which solid forms are characterized involves analyzing data collected on the polymorphic forms so as to allow one of ordinary skill in the art to distinguish one solid form from other solid forms containing the same material. Chemical identity of solid forms can often be determined with solution-state techniques such as ¹³C NMR or ¹H NMR. While these may help identify a material, and a solvent molecule for a solvate, such solution-state techniques themselves may not provide information about the solid state. There are, however, solid-state analytical techniques that can be used to provide information about solid-state structure and differentiate among polymorphic solid forms, such as single crystal X-ray diffraction, X-ray powder diffraction (XRPD), solid state nuclear magnetic resonance (SS-NMR), and infrared and Raman spectroscopy, and thermal techniques such as differential scanning calorimetry (DSC), thermogravimetry (TG), melting point, and hot stage microscopy.

To “characterize” a solid form of a compound, one may, for example, collect XRPD data on solid forms of the compound and compare the XRPD peaks of the forms. For example, when only two solid forms, I and II, are compared and the form I pattern shows a peak at an angle where no peaks appear in the form II pattern, then that peak, for that compound, distinguishes form I from form II and further acts to characterize form I. The collection of peaks which distinguish form I from the other known forms is a collection of peaks which may be used to characterize form I. Those of ordinary skill in the art will recognize that there are often multiple ways, including multiple ways using the same analytical technique, to characterize solid forms. Additional peaks could also be used, but are not necessary, to characterize the form up to and including an entire diffraction pattern. Although all the peaks within an entire XRPD pattern may be used to characterize such a form, a subset of that data may, and typically is, used to characterize the form.

An XRPD pattern is an x-y graph with diffraction angle (typically ° 2θ) on the x-axis and intensity on the y-axis. The peaks within this pattern may be used to characterize a crystalline solid form. As with any data measurement, there is variability in XRPD data. The data are often represented solely by the diffraction angle of the peaks rather than including the intensity of the peaks because peak intensity can be particularly sensitive to sample preparation (for example, particle size, moisture content, solvent content, and preferred orientation effects influence the sensitivity), so samples of the same material prepared under different conditions may yield slightly different patterns; this variability is usually greater than the variability in diffraction angles. Diffraction angle variability may also be sensitive to sample preparation. Other sources of variability come from instrument parameters and processing of the raw X-ray data: different X-ray instruments operate using different parameters and these may lead to slightly different XRPD patterns from the same solid form, and similarly different software packages process X-ray data differently and this also leads to variability. These and other sources of variability are known to those of ordinary skill in the pharmaceutical arts. Due to such sources of variability, it is usual to assign a variability of ±0.2° 2θ to diffraction angles in XRPD patterns.

“Comprising” or “comprises” is intended to mean that the compositions and methods include the recited elements, but not exclude others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this invention.

The term “does not undergo polymorphic transformation” implies that form I, when exposed as a slurry and allowed to equilibrate with forms II and III returns to form I. Similarly forms II and III, when treated as slurries, convert to form I. On prolonged storage form I remains stable.

“Room temperature” refers to (22±5) ° C.

“Storing” or “storage” refers to storing crystalline Compound 1 hydrochloride ansolvate Form I or a composition including the Form I such that no more than about 10%, more preferably no more than about 5%, still more preferably no more than about 3%, or most preferably no more than about 1% of the ansolvate Form I undergoes transformation to another Form, e.g., Form II or III.

“Therapeutically effective amount” or “therapeutic amount” refers to an amount of a drug or an agent that when administered to a patient suffering from a condition, will have the intended therapeutic effect, e.g., alleviation, amelioration, palliation or elimination of one or more manifestations of the condition in the patient. The therapeutically effective amount will vary depending upon the subject and the condition being treated, the weight and age of the subject, the severity of the condition, the particular composition or excipient chosen, the dosing regimen to be followed, timing of administration, the manner of administration and the like, all of which can be determined readily by one of ordinary skill in the art. The full therapeutic effect does not necessarily occur by administration of one dose, and may occur only after administration of a series of doses. Thus, a therapeutically effective amount may be administered in one or more administrations. For example, and without limitation, a therapeutically effective amount of an agent, in the context of treating disorders related to hemoglobin S, refers to an amount of the agent that alleviates, ameliorates, palliates, or eliminates one or more manifestations of the disorders related to hemoglobin S in the patient.

“Treatment”, “treating”, and “treat” are defined as acting upon a disease, disorder, or condition with an agent to reduce or ameliorate the harmful or any other undesired effects of the disease, disorder, or condition and/or its symptoms. Treatment, as used herein, covers the treatment of a human patient, and includes: (a) reducing the risk of occurrence of the condition in a patient determined to be predisposed to the disease but not yet diagnosed as having the condition, (b) impeding the development of the condition, and/or (c) relieving the condition, i.e., causing regression of the condition and/or relieving one or more symptoms of the condition. For purposes of this invention, beneficial or desired clinical results include, but are not limited to, multilineage hematologic improvement, decrease in the number of required blood transfusions, decrease in infections, decreased bleeding, and the like.

### Identifying the Ansolvate Form I

A solid form screen was carried out in 96-well format on Compound 1 hydrochloride. Once solid samples were harvested from crystallization attempts, they were examined under a microscope for birefringence and morphology. The solid samples were characterized by various techniques including those described herein. A number of different crystallization techniques were used as set forth herein.

Fast evaporation: solutions are prepared in various solvents and sonicated between aliquot additions to assist in dissolution. Once a mixture reached complete dissolution, as judged by visual observation, the solution was filtered through a 0.2 μm nylon filter. The filtered solution was allowed to evaporate at room temperature in an open vial, and the solids that formed were isolated by filtration and dried.

Slow evaporation: solutions are prepared as for the fast evaporation technique above, and the filtered solution was allowed to evaporate at room temperature in a vial covered with aluminum foil perforated with pinholes. The solids that formed are isolated by filtration and dried.

Slow cooling: saturated solutions are prepared in various solvents at elevated temperatures and filtered through a 0.2 μm nylon filter into an open vial while still warm. The vial is covered and allowed to cool slowly to room temperature, and the presence or absence of solids is noted. If there are no solids present, or if the amount of solids was judged too small for XRPD analysis, the vial is placed in a refrigerator overnight. Again, the presence or absence of solids is noted and if there are none, the vial is placed in a freezer overnight. Solids that form are isolated by filtration and dried.

Crash cooling: saturated solutions are prepared in various solvents or solvent systems at an elevated temperature and filtered through a 0.2 μm nylon filter into an open vial while still warm. The vial is covered and placed directly into a freezer. The presence or absence of solids is noted. Solids that form are isolated by filtration and dried.

Antisolvent crystallization: solutions are prepared in various solvents at elevated temperature and filtered through a 0.2 μm nylon filter. Solid formation is induced by adding the filtered solution to an appropriate anti-solvent at a temperature below room temperature. The resulting solids are isolated by filtration and dried.

Slurrying: slurries are prepared by adding enough solids to a given solvent so that undissolved solids are present. The mixture is then agitated in a sealed vial at a chosen temperature. After time, the solids are isolated by filtration and dried.

Stress experiments: solids are stressed under different temperature and/or relative humidity (RH) environments for a measured time period. Specific RH values are achieved by placing the sample inside sealed chambers optionally containing saturated salt solutions. Samples are analyzed by XRPD immediately after removal from the stress environment.

In addition to form I, two additional solid forms II and III were identified. Of the three additional forms, only one, Form I, was confirmed to have an unsolvated structure, crystalline 2-hydroxy-6-((2-(1-isopropyl-1H-pyrazol-5-yl)pyridin-3-yl)methoxy)benzaldehyde hydrochloride ansolvate.

### Stable Ansolvate Form I and its Properties

In one embodiment, this invention provides a crystalline Compound 1 salt ansolvate and, in particular, the hydrochloride ansolvate (crystalline Form I). In another embodiment, this invention provides a composition comprising the crystalline Compound 1 hydrochloride ansolvate. Preferably, the crystalline Form I is substantially free of a solvated polymorph of Compound 1 hydrochloride. “Substantially free” of a solvated polymorph of Compound 1 hydrochloride refers to a crystalline Form I, which excludes solvated polymorph of Compound 1 hydrochloride to an extent that the Form I crystals are suitable for human administration. In one embodiment, the crystalline Form I contains up to about 5%, more preferably about 3%, and still more preferably about 1% of one or more solvated polymorph of Compound 1 hydrochloride. In one embodiment, the less stable (than Form I) polymorph is a form II or form III polymorph. As used herein, solvate includes a hydrate form as well.

It is possible to attain the ansolvate Form I with such high polymorphic purity due, in part, to the surprising stability of the ansolvate, and its resistance to conversion to another form such as Forms II and III. In contrast, the polymorph Form II transforms almost entirely to Form I crystals during interconversion experiments described herein.

### Pharmaceutical Compositions

In another of its composition embodiments, this invention provides for a pharmaceutical composition comprising a pharmaceutically acceptable excipient and crystalline Compound 1 ansolvate salt, such as the hydrochloride Form I.

Such compositions can be formulated for different routes of administration. Although compositions suitable for oral delivery will probably be used most frequently, other routes that may be used include intravenous, intraarterial, pulmonary, rectal, nasal, vaginal, lingual, intramuscular, intraperitoneal, intracutaneous, intracranial, subcutaneous and transdermal routes. Suitable dosage forms for administering any of the compounds described herein include tablets, capsules, pills, powders, aerosols, suppositories, parenterals, and oral liquids, including suspensions, solutions and emulsions. Sustained release dosage forms may also be used, for example, in a transdermal patch form. All dosage forms may be prepared using methods that are standard in the art (see e.g., Remington's Pharmaceutical Sciences, 16^{th} ed., A. Oslo editor, Easton Pa. 1980).

Pharmaceutically acceptable excipients are non-toxic, aid administration, and do not adversely affect the therapeutic benefit of the compound of this invention. Such excipients may be any solid, liquid, semi-solid or, in the case of an aerosol composition, gaseous excipient that is generally available to one of skill in the art. Pharmaceutical compositions in accordance with the invention are prepared by conventional means using methods known in the art.

The compositions disclosed herein may be used in conjunction with any of the vehicles and excipients commonly employed in pharmaceutical preparations, e.g., talc, gum arabic, lactose, starch, magnesium stearate, cocoa butter, aqueous or non-aqueous solvents, oils, paraffin derivatives, glycols, etc. Coloring and flavoring agents may also be added to preparations, particularly to those for oral administration. Solutions can be prepared using water or physiologically compatible organic solvents such as ethanol, 1,2-propylene glycol, polyglycols, dimethylsulfoxide, fatty alcohols, triglycerides, partial esters of glycerin and the like.

Solid pharmaceutical excipients include starch, cellulose, hydroxypropyl cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. In certain embodiments, the compositions provided herein comprises one or more of a-tocopherol, gum arabic, and/or hydroxypropyl cellulose.

In one embodiment, this invention provides sustained release formulations such as drug depots or patches comprising an effective amount of a compound provided herein. In another embodiment, the patch further comprises gum Arabic or hydroxypropyl cellulose separately or in combination, in the presence of alpha-tocopherol. Preferably, the hydroxypropyl cellulose has an average MW of from 10,000 to 100,000. In a more preferred embodiment, the hydroxypropyl cellulose has an average MW of from 5,000 to 50,000.

Compounds and pharmaceutical compositions of this invention maybe used alone or in combination with other compounds. When administered with another agent, the co-administration can be in any manner in which the pharmacological effects of both are manifest in the patient at the same time. Thus, co-administration does not require that a single pharmaceutical composition, the same dosage form, or even the same route of administration be used for administration of both the compound of this invention and the other agent or that the two agents be administered at precisely the same time. However, co-administration will be accomplished most conveniently by the same dosage form and the same route of administration, at substantially the same time. Obviously, such administration most advantageously proceeds by delivering both active ingredients simultaneously in a novel pharmaceutical composition in accordance with the present invention.

### Treatment Methods

In another aspect, the present invention provides a method of preparing the solid crystalline Compound 1 ansolvate salt. In one embodiment, the ansolvate salt is a hydrochloride ansolvate Form I. In one embodiment, provided herein is a method of preparing a solid crystalline hydrochloride ansolvate of Form I comprising slurrying or contacting a hydrochloride of the Compound 1 in or with methyl ethyl ketone (or 2-butanone) at room temperature. Form I can also be prepared under same or similar conditions from methanol, ethanol, 2-propanol, 2-methyl-1-propanol, 1-butanol, tetrahydrofuran; or a binary solvent combination of methanol with ethanol, 2-propanol, 2-methyl-1-propanol, 1-butanol, 2-butanone, tetrahydrofuran, acetonitrile, METB, acetone, isopropyl acetate, ethyl acetate, toluene; or a binary solvent combination of ethanol with 2-propanol, 2-methyl-1-propanol, 2-butanone, tetrahydrofuran, METB, acetone, isopropyl acetate, or ethyl acetate. at room temperature.

In yet another of its method embodiments, there are provided methods for increasing oxygen affinity of hemoglobin S in a subject, the method comprising administering to a subject in need thereof a therapeutically effective amount of a crystalline ansolvate salt of Compound 1, such as the hydrochloride salt of Form I.

In yet another of its method embodiments, there are provided methods for treating oxygen deficiency associated with sickle cell anemia in a subject, the method comprising administering to a subject in need thereof a therapeutically effective amount of a crystalline ansolvate salt of Compound 1, such as the hydrochloride salt of Form I.

In further aspects of the invention, a method is provided for treating sickle cell disease, the method comprising administering to a subject in need thereof a therapeutically effective amount of crystalline Compound 1, such as the hydrochloride salt of Form I. In still further aspects of the invention, a method is provided for treating cancer, a pulmonary disorder, stroke, high altitude sickness, an ulcer, a pressure sore, Alzheimer's disease, acute respiratory disease syndrome, and a wound, the method comprising administering to a subject in need thereof a therapeutically effective amount of crystalline Compound 1, such as the hydrochloride salt of Form I ansolvate salt.

In such treatments, the dosing of the crystalline Compound 1, such as the hydrochloride salt of Form I ansolvate salt, to the treated patient is already disclosed in the art.

In one embodiment of any of the compositions and methods described herein, the ansolvate salt is a hydrochloride ansolvate Form I. In other embodiments, the salt is the hydrobromide or a hemi H₂SO₄ salt, such as that containing 2 moles of Compound 1 per mole of H₂SO₄ or an H₂SO₄ salt.

### EXAMPLES

The following examples describe the preparation, characterization, and properties of the hydrochloride salt of Compound 1 Form I ansolvate. Unless otherwise stated, all temperatures are in degrees Celcius (° C.) and the following abbreviations have the following definitions:
DSC Differential scanning calorimetry
DVS Dynamic vapor sorption
HPLC High performance liquid chromatography
NA Not applicable
ND Not determined
Q Percent dissolved per unit time
RH Relative humidity
RSD Residual standard deviation
RRT Relative retention time
SS-NMR Solid state nuclear magnetic resonance
TGA Thermogravimetric analysis
TG-IR Thermogravimetric infra red analysis
XRPD X-ray powder diffraction
VT-XRPD Variable temperature X-ray powder diffraction

The compound of formula (I) was synthesized as schematically described below and elaborated thereafter.

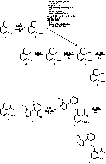

### Example 1

### Synthesis of Compound 15

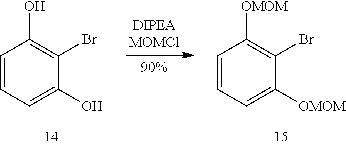

To a solution of 2-bromobenzene-1,3-diol (5 g, 26.45 mmol) DCM (50 ml) at 0° C. was added DIPEA (11.54 mL, 66.13 mmol) and MOMCl (4.42 mL, 58.19 mmol). The mixture was stirred at 0° C. for 1.5 h, and then warmed to room temperature. The solution was diluted with DCM, washed with sat. NaHCO₃, brine, dried and concentrated to give crude product, which was purified by column (hexanes/EtOAc=4:1) to give desired product 15.58 g (90%).

### Example 2

### Synthesis of Compound 13 from 15

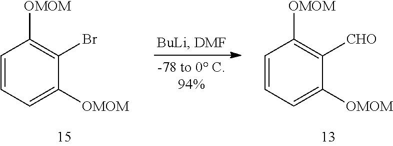

To a solution of 2-bromo-1,3-bis(methoxymethoxy)benzene (15) (19.9 g, 71.8 mmol) in THF (150 mL) at −78° C. was added BuLi (2.5 M, 31.6 mL, 79.0 mmol) dropwise. The solution was stirred at −78° C. for 25 min (resulting white cloudy mixture), then it was warmed to 0° C. and stirred for 25 min. The reaction mixture slowly turns homogenous. To the solution was added DMF at 0° C. After 25 min, HPLC showed reaction completed. The mixture was quenched with sat. NH4Cl (150 mL), diluted with ether (300 mL). The organic layer was separated, aq layer was further extracted with ether (2×200 mL), and organic layer was combined, washed with brine, dried and concentrated to give crude product, which was triturated to give 14.6 g desired product. The filtrate was then concentrated and purified by column to give additional 0.7 g, total mass is 15.3 g.

### Example 3

### Synthesis of Compound 13 from Resorcinol 11

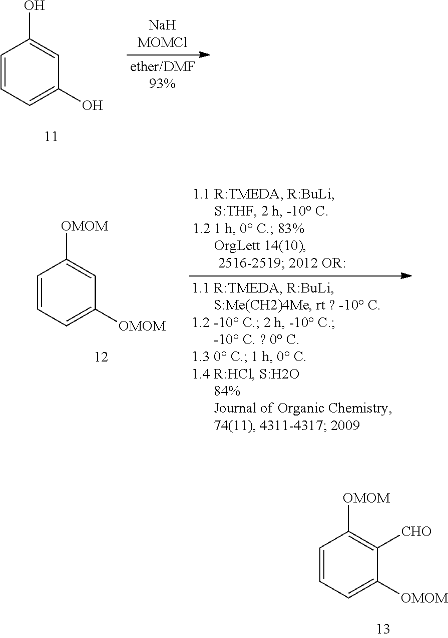

A three-necked round-bottom flask equipped with mechanical stirrer was charged with 0.22 mol of NaH (50% suspension in mineral oil) under nitrogen atmosphere. NaH was washed with 2 portions (100 mL) of n-hexane and then with 300 mL of dry diethyl ether; then 80 mL of anhydrous DMF was added. Then 0.09 mol of resorcinol 11, dissolved in 100 mL of diethyl ether was added dropwise and the mixture was left under stirring at rt for 30 min. Then 0.18 mol of MOMCl was slowly added. After 1 h under stirring at rt, 250 mL of water was added and the organic layer was extracted with diethyl ether. The extracts were washed with brine, dried (Na₂SO₄), then concentrated to give the crude product that was purified by silica gel chromatography to give compound 12 (93% yield).

A three-necked round-bottom flask was charged with 110 mL of n-hexane, 0.79 mol of BuLi and 9.4 mL of tetramethylethylendiamine (TMEDA) under nitrogen atmosphere. The mixture was cooled at −10° C. and 0.079 mol of bis-phenyl ether 12 was slowly added. The resulting mixture was left under magnetic stirring at −10° C. for 2 h. Then the temperature was raised to 0° C. and 0.067 mol of DMF was added dropwise. After 1 h, aqueous HCl was added until the pH was acidic; the mixture was then extracted with ethyl ether. The combined extracts were washed with brine, dried (Na₂SO₄), and concentrated to give aldehyde 13 (84%).

2,6-bis(methoxymethoxy)benzaldehyde (13): mp 58-59° C. (n-hexane); IR (KBr) n: 1685 (C═O) cm⁻¹; ¹H-NMR (400 MHz, CDCl₃) δ 3.51 (s, 6H, 2 OCH₃), 5.28 (s, 4H, 2 OCH₂O), 6.84 (d, 2H, J=8.40 Hz, H-3, H-5), 7.41 (t, 1H, J=8.40 Hz, H-4), 10.55 (s, 1H, CHO); MS, m/e (relative intensity) 226 (M+, 3), 180 (4), 164 (14), 122 (2), 92 (2), 45 (100); Anal. Calc'd. for C₁₁H₁₄O₅: C,58.40; H, 6.24. Found: C, 57.98; H, 6.20.

### Example 4

### The Synthesis of Compound 16

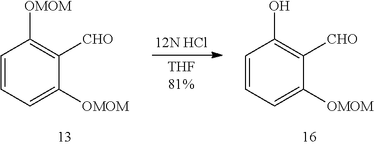

To a solution of 2,6-bis(methoxymethoxy)benzaldehyde (13) (15.3 g, 67.6 mmol) in THF (105 mL) (solvent was purged with N₂) was added conc. HCl (12N, 7 mL) under N₂, then it was further stirred under N₂ for 1.5 h. To the solution was added brine (100 mL) and ether (150 ml). The organic layer was separated and the aqueous layer was further extracted with ether (2×200 mL). The organic layer was combined, washed with brine, dried and concentrated to give crude product, which was purified by column (300 g, hexanes/EtOAc=85:15) to give desired product 16 (9.9 g) as yellow liquid.

### Example 5

### Synthesis of Compound 17

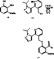

To a solution of 2-hydroxy-6-(methoxymethoxy)benzaldehyde (16) (10.88 g, 59.72 mmol) in DMF (120 mL) (DMF solution was purged with N₂ for 10 min) was added K₂CO₃ (32.05 g, 231.92 mmol) and 3-(chloromethyl)-2-(1-isopropyl-1H-pyrazol-5-yl)pyridine hydrochloride (10) (15.78 g, 57.98 mmol). The mixture was heated at 65° C. for 1.5 h, cooled to rt, poured into ice water (800 mL). The precipitated solids were isolated by filtration, dried and concentrated to give desired product (17, 18 g).

### Example 6

### Synthesis of Compound (I)

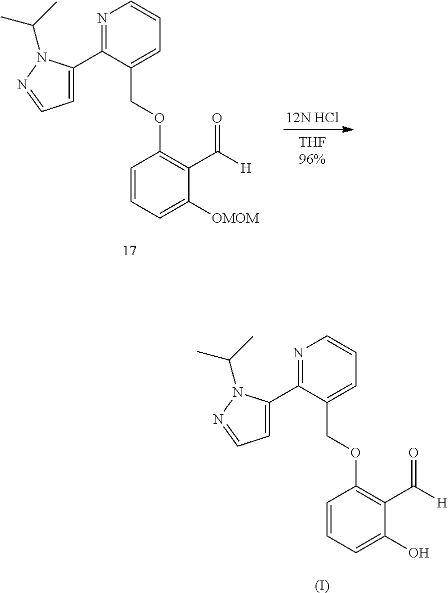

To a solution of 2-((2-(1-isopropyl-1H-pyrazol-5-yl)pyridin-3-yl)methoxy)-6-(methoxymethoxy)benzaldehyde (17) (18 g, 47.19 mmol) in THF (135 mL, solution was purged with N₂) was added conc. HCl (12N, 20 mL). The solution was stirred at rt for 3 h when HPLC showed the reaction complete. The mixture was added to a solution of NaHCO₃ (15 g) in water (1.2 L), and the resulting precipitate was collected by filtration, dried to give crude solid, which was further purified by column (DCM/EtOAc=60:40) to give pure product (15.3 g).

### Example 7

### Synthesis of Compound I (Free Base) and its HCl Salt Form

Compound (I) free base (40 g) was obtained from the coupling of the alcohol intermediate 7 and 2,6-dihydroxybenzaldedhye 9 under Mitsunobu conditions. A procedure is also provided below:

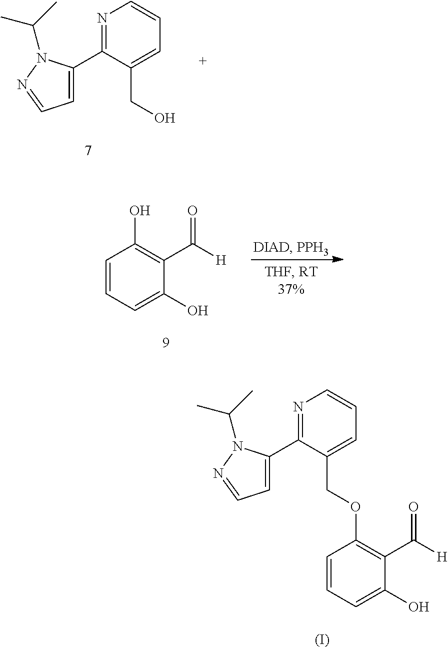

### Example 8

### Synthesis of Compound (I) by Mitsunobu Coupling

Into a 2000-mL three neck round-bottom flask, which was purged and maintained with an inert atmosphere of nitrogen, was placed a solution of [2-[1-(propan-2-yl)-1H-pyrazol-5-yl]pyridin-3-yl]methanol (7) (70 g, 322.18 mmol, 1.00 equiv) in tetrahydrofuran (1000 mL). 2,6-Dihydroxybenzaldehyde (9) (49.2 g, 356.21 mmol, 1.10 equiv) and PPh₃ (101 g, 385.07 mmol, 1.20 equiv) were added to the reaction mixture. This was followed by the addition of a solution of DIAD (78.1 g, 386.23 mmol, 1.20 equiv) in tetrahydrofuran (200 ml) dropwise with stirring. The resulting solution was stirred overnight at room temperature. The resulting solution was diluted with 500 ml of H₂O. The resulting solution was extracted with 3×500 ml of dichloromethane and the combined organic layers were dried over sodium sulfate and concentrated under vacuum. The residue was applied onto a silica gel column with EA:PE (1:50-1:3) as eluent to yield the crude product. The crude product was re-crystallized from i-propanol/H₂O in the ratio of 1/1.5. This resulted in 40 g (37%) of 2-hydroxy-6-([2-[1-(propan-2-yl)-1H-pyrazol-5-yl]pyridin-3-yl]methoxy)benzaldehyde as a light yellow solid. The compound exhibited a melting point of 80-82° C. MS (ES, m/z): 338.1 [M+1]. ¹H NMR (300 MHz, DMSO-d6) δ 11.72(s, 1H), 10.21(s, 1H), 8.76(d, J=3.6 Hz, 1H), 8.24(d, J=2.7 Hz, 1H),7.55(m, 3H), 6.55(m,3H) ,5.21 (s, 2H), 4.65 (m, 1H), 1.37 (d, J=5.1 Hz, 6H). ¹H NMR (400 MHz, CDCl₃) δ 11.96 (s, 1H), 10.40 (s, 1H), 8.77 (dd, J=4.8, 1.5 Hz, 1H), 8.00 (d, J=7.8 Hz, 1H), 7.63 (d, J=1.8 Hz, 1H), 7.49-7.34 (m, 2H), 6.59 (d, J=8.5 Hz, 1H), 6.37 (d, J=1.8 Hz, 1H), 6.29 (d, J=8.2 Hz, 1H), 5.10 (s, 2H), 4.67 (sep, J=6.7 Hz, 1H), 1.50 (d, J=6.6 Hz, 6H).

In another approach, multiple batches of Compound (I) free base are prepared in multi gram quantities (20 g). The advantage of this route is the use of mono-protected 2,6-dihydroxybenzaldehyde (16), which effectively eliminates the possibility of bis-alkylation side product. The mono-MOM ether of 2,6-dihydroxybenzaldehyde (16) can be obtained from two starting points, bromoresorcinol (14) or resorcinol (11) [procedures described in the Journal of Organic Chemistry, 74(11), 4311-4317; 2009 ]. All steps and procedures are provided below. Due to the presence of phenolic aldehyde group, precautions (i.e., carry out all reactions under inert gas such as nitrogen) should be taken to avoid oxidation of the phenol and/or aldehyde group.

Preparation of compound I HCl salt: A solution of compound I (55.79 g, 165.55 mmol) in acetonitrile (275 mL) was flushed with nitrogen for 10 min, then to this solution was added 3N aqueous HCl (62 mL) at room temperature. The mixture was stirred for additional 10 min after the addition, most of the acetonitrile (about 200 mL) was then removed by evaporation on a rotary evaporator at around 32° C., the remaining solution was frozen by cooling in an acetone-dry ice bath and lyophilized to afford compound I HCl salt (59.4 g).

### Example 9

### Crystallizations of 2-hydroxy-6-((2-(1-isopropyl-1H-pyrazol-5-yl)pyridin-3-yl)methoxy)benzaldehyde hydrochloride

Methods: Three crystal forms of 2-hydroxy-6-((2-(1-isopropyl-1H-pyrazol-5-yl)pyridin-3-yl)methoxy)benzaldehyde hydrochloride with different XRPD patterns were identified in this study based on a matrix of crystallizations in various solvents, screened in in 96-wells as described below. An initial drug substance was assigned as Form I. Form I was also obtained in numerous conditions during solvent crystallization in the 96-well screen and in slurry studies. Form II and Form III were also generated. Solids obtained were characterized by X-ray powder diffraction (XRPD), differential scanning calorimetry (DSC), thermogravimetry (TGA), polarizing microscope (PLM) and Fourier transform infrared spectrometer (FTIR).

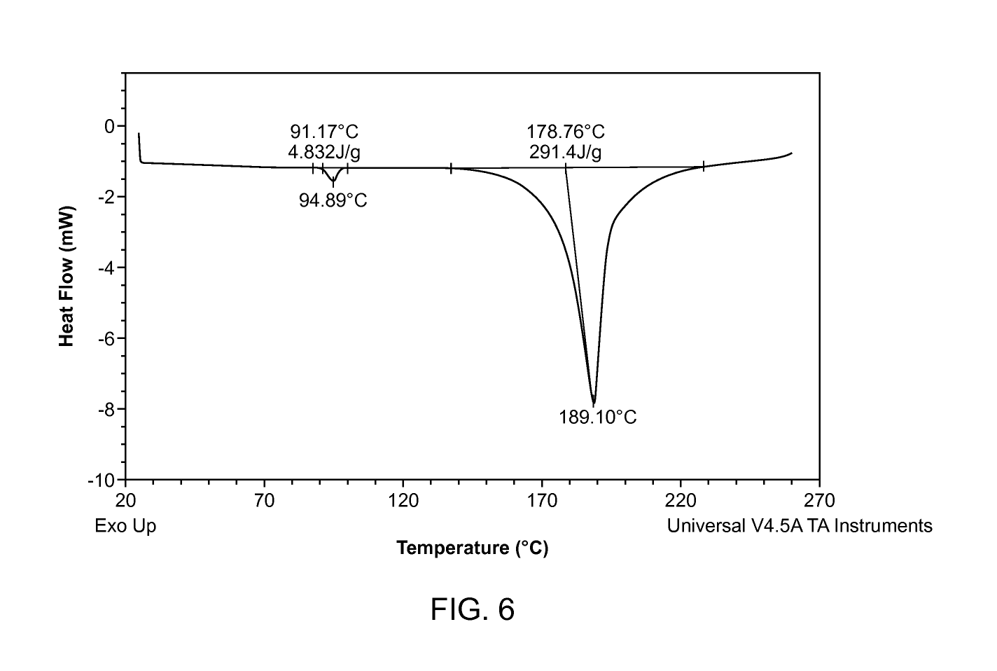

Some solid samples from slurry experiments showed almost identical XRPD pattern as Form I except one peak at 20 angle at 18 degrees. However, the marginal difference in XRPD of those samples may be due to the preferred orientation possibly caused by particle size and morphology differences. Form II and Form III were unstable at ambient conditions and converted into Form I or a mixture of Form I and its free base. In an interconversion study, a mixture of all identified crystal forms turned into Form I after slurry experiments in ethyl acetate or ethanol. The result suggests that Form I is the most thermodynamically stable form. The XRPD patterns and microscopy images of the three crystal forms are shown in FIG. 6 and FIG. 7, respectively.

Powder X-RayDiffraction (XRPD): The solid samples were examined using X-ray diffractometer (Bruker D8 advance). The system is equipped with LynxEye detector. The samples were scanned from 3 to 40° 2θ, at a step size 0.02° 2θ. The tube voltage and current were 40 KV and 40 mA respectively. The sample was transferred from sample container onto zero background XRD holder and gently ground.

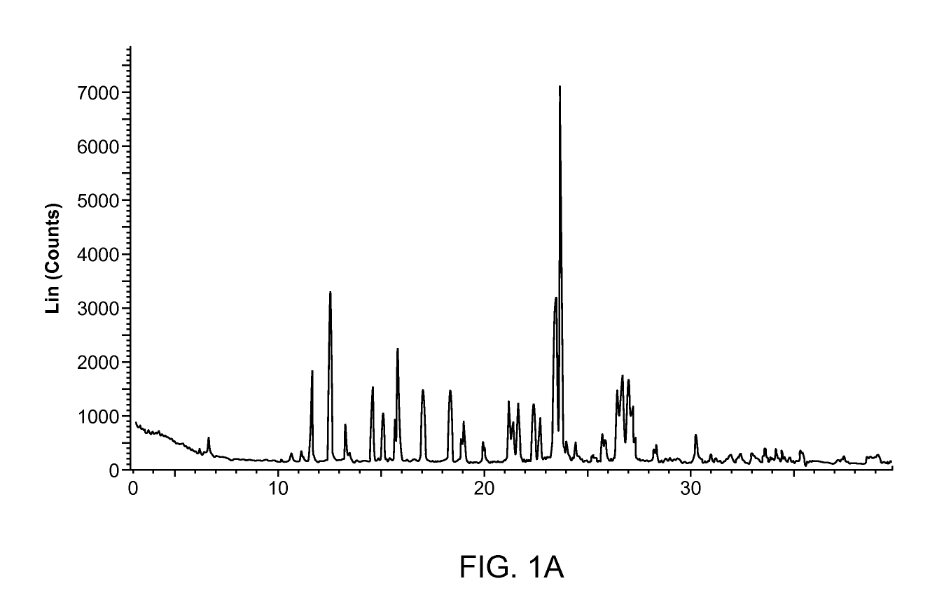

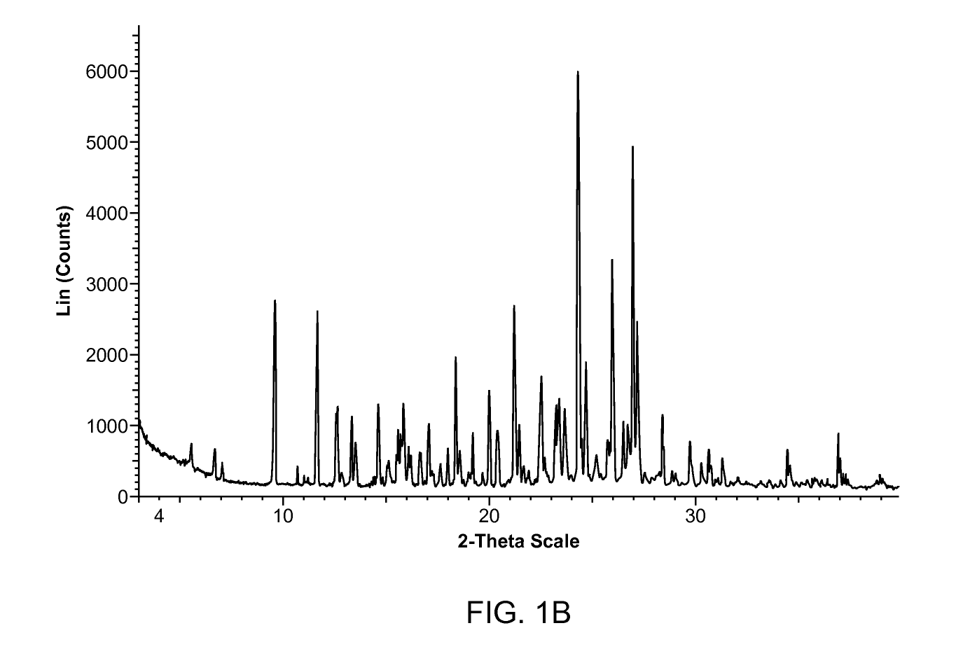

TABLE 1
XRPD Pattern Peak List of Form I of Compound
1 hydrochloride (See FIG. 1A)

| Pos. [°2 θ] | Rel. Int. [%] |
|---|---|
| 11.6 | 26.1 |
| 12.6 | 47.2 |
| 13.3 | 12.0 |
| 14.6 | 22.5 |
| 15.1 | 15.2 |
| 15.8 | 33.3 |
| 17.1 | 20.8 |
| 18.4 | 21.5 |
| 19.0 | 11.7 |
| 21.2 | 17.3 |
| 21.4 | 11.7 |
| 21.7 | 17.0 |
| 22.4 | 17.2 |
| 22.7 | 12.7 |
| 23.4 | 45.3 |
| 23.7 | 100.0 |
| 26.5 | 20.7 |
| 26.7 | 24.3 |
| 27.0 | 22.9 |
| 27.2 | 15.6 |

TABLE 2
XRPD Pattern Peak List of Form II of Compound
1 hydrochloride (See FIG. 1B)

| Pos. [°2 θ] | Rel. Int. [%] |
|---|---|
| 9.6 | 54.6 |
| 11.6 | 51.7 |
| 12.6 | 22.6 |
| 13.3 | 22.0 |
| 14.6 | 26.0 |
| 15.8 | 25.2 |
| 17.1 | 20.1 |
| 18.4 | 38.5 |
| 20.0 | 28.8 |
| 21.2 | 48.6 |
| 22.5 | 33.5 |
| 23.4 | 25.9 |
| 23.7 | 23.7 |
| 24.3 | 100.0 |
| 24.7 | 37.3 |
| 26.0 | 65.7 |
| 26.5 | 20.9 |
| 27.0 | 97.3 |
| 27.2 | 43.1 |
| 28.4 | 22.4 |

TGA Analysis: TGA analysis was carried out on a TA Instruments TGA Q500. Samples was placed in a tarred platinum or aluminum pan, automatically weighed and inserted into the TGA furnace. The samples were heated at a rate of 10° C./min to final temperature of 300° C. The purge gas is nitrogen for balance at 40 ml/min and for the sample at 60 ml/min, respectively.

DSC Analysis: DSC analysis was conducted on a TA Instruments Q200. The calibration standard was indium. A sample weighed was placed into a TA DSC pan with cover, and weight was accurately recorded. Crimped pans were used for analysis and the samples were heated under nitrogen (50 ml/min), firstly equilibrate at 25° C. and then up to a final temperature of 270° C. at a rate of 10° C./min.

Polarizing Microscopy Analysis: PLM analysis was conducted on a Nikon Instruments Eclipse 80i. The images were captured by a DS camera and transmitted to the computer. The photos were processed using the NIS-Elements D3.0 software.

Dynamic Vapor Sorption (DVS): Dynamic moisture adsorption and desorption was studied using IGA Sorp (Hidden Isochema Ltd.Warrington, UK). About 5 mg (3˜5 mg) of a prepared sample was placed in a sample basket and hung in the measuring chamber of an IGA Sorp. For an isotherm test, the chamber temperature is maintained by a water bath at constant 25.0±1.0° C. The sample was tested at a targeted RH range from 0% to 90% full cycle in step mode. The analysis is performed in 10% increment. Time duration at each RH was set as min. 30

Fourier Transform Infrared Spectometry (FTIR): FTIR analysis was carried out on instrument SHIMADZU FTIR-84005. The sample was pressed into tablet together with KBr firstly and then performed according to SOP of IR.

### Example 10

### 96-Well Plate Screening

Preparation of Drug Solutions: 60 mg of 2-hydroxy-6-((2-(1-isopropyl-1H-pyrazol-5-yl)pyridin-3-yl)methoxy)benzaldehyde hydrochloride was dissolved in 3.0 mL of various pairs of solvents selected from methanol, ethanol, 2-propanol, 2-methyl-1-propanol, 1-butanol, 2-butanone, THF, acetonitrile, MTBE, acetone, isopropyl acetate, ethyl acetate and toluene. 2 mL of drug solutions/suspensions above were manually filtered into clean glass vials using plastic non-contaminating syringes equipped with 0.22 μm nylon filter cartridges and then all filtrates were collected used for crystallization studies as outlined below. Remaining suspensions consisting of solid particulates were afterward used for slurry studies.

Well Plate Preparation: The saturated drug solutions (filtrates) were distributed in 96-well plate, according to to solvent matrix drawn from methanol, ethanol, 2-propanol, 2-methyl-1-propanol, 1-butanol, 2-butanone, THF, acetonitrile, MTBE, acetone, isopropyl acetate, ethyl acetate and toluene. 100 uL of each filtrate above was mixed with the same amount of another one in a well (total 91 wells). The plate was covered with a film with pin holes and then the solutions were allowed to evaporate in an operating laboratory fume hood under ambient condition (temperature and humidity). During the process of crystallization, the plate was visually examined and any solid material was analyzed by imaging system, XRPD, DSC and TGA based on the amount of samples obtained.

Solvent Crystallization: Remaining filtrates after well plate preparation were allowed to evaporate to dryness in glass test tubes in an operating laboratory fume hood under ambient conditions. Any solids will be analyzed by XRPD, DSC, TGA and Microscopy, as deemed appropriate based on amount of sample.

Slurry Study: Remaining suspensions were allowed to keep stirring in an operating laboratory fume hood under ambient conditions (temperature & humidity). After 3 days, the suspensions were filtered and the solids were collected. These solids were analyzed by XRPD, DSC, TGA and Microscopy based on amount of samples.

Interconversion Study: Interconversion experiments were carried out by making slurries containing the same amount of different forms solids in ethyl acetate or ethanol. The slurries were kept stirring for days at ambient condition. The insoluble solids were recovered by filtration and examined using XRPD.

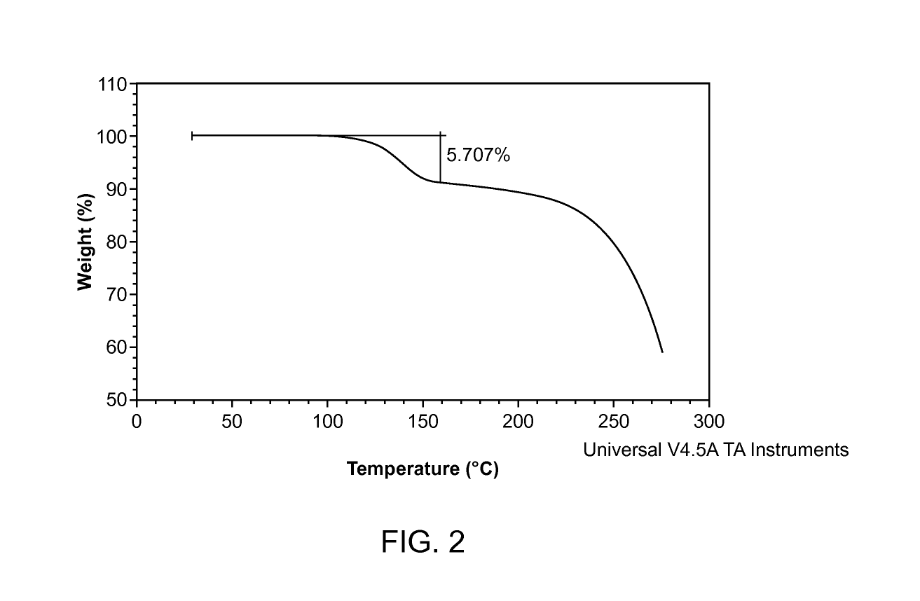

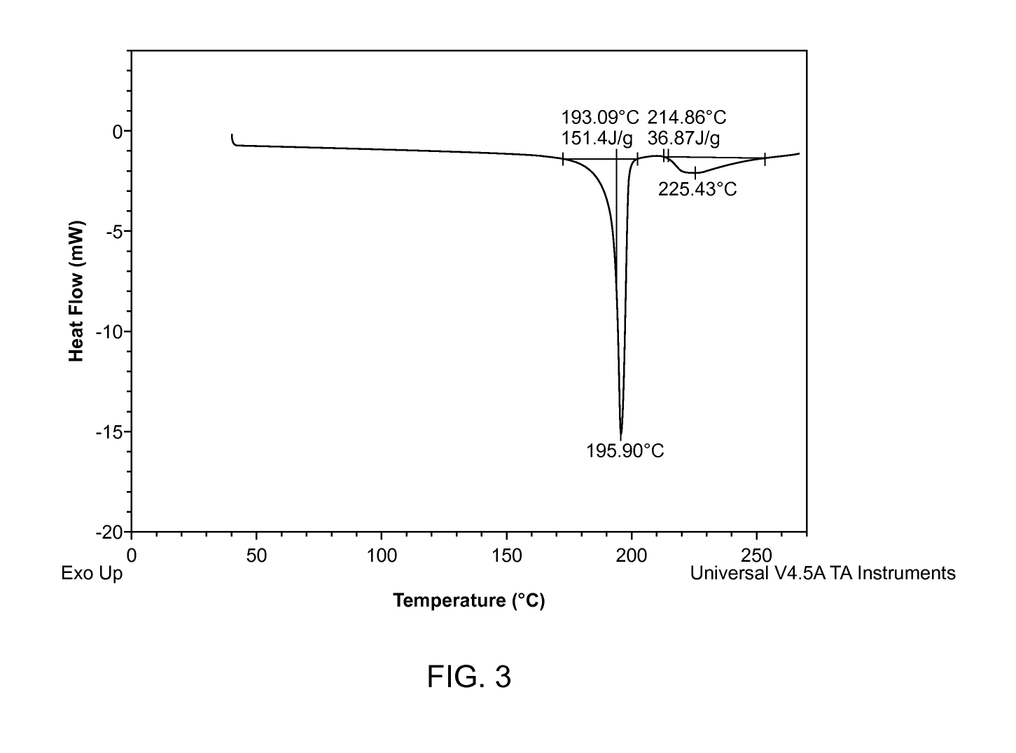

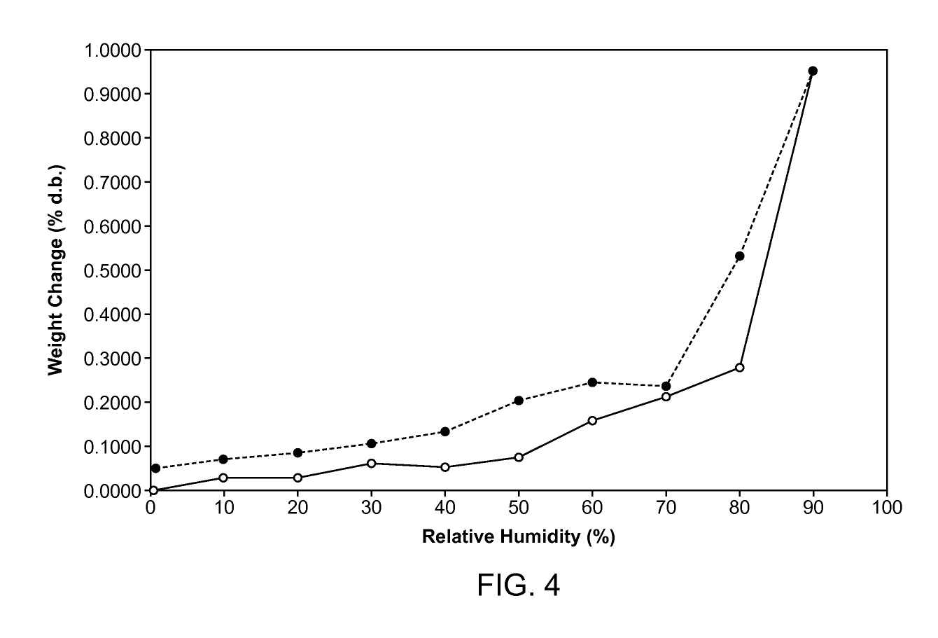

Solid State Characterizaton: Form I of Compound 1 hydrochloride was characterized by XRPD (FIG. 1), TGA (FIG. 2), DSC (FIG. 3), DVS (FIG. 4). XRPD and Microscopy showed that the drug substance was crystalline. TGA profile showed a weight loss of 8.71% prior to decomposition, which was resulted from the loss of HCl. DSC profile showed that there were one main endothermic melting peak with peak temperature of 195.9° C. and enthalpy of 151.4 J/g. DVS profile showed that it only picks up <1% water at 80% RH. The crystal form of the initial drug substance is assigned as Form I.

### Example 11

### Scale-Up of Form I

To a solution of Compound 1 (150 g, 443.8 mmol) in methyl ethyl ketone (750 mL) was added, drop-wise, concentrated hydrochloric acid (67.5 g, 665.7 mmol, 1.5 eq) at 30+5° C. After the addition, the mixture was cooled and kept at 0±5° C. for 1 hr. Crystals were collected by filtration, washed with methyl ethyl ketone (300 mL) and then dried in a vacuum oven at 30±5° C. to give 140 g (84%) of the HCl salt Form I as a light yellow solid.

### Example 12

### Scale-Up of Forms II and III

Based on the initial 96-well plate screening results, solvent crystallization and slurry studies, Form II and Form III were further scaled up.

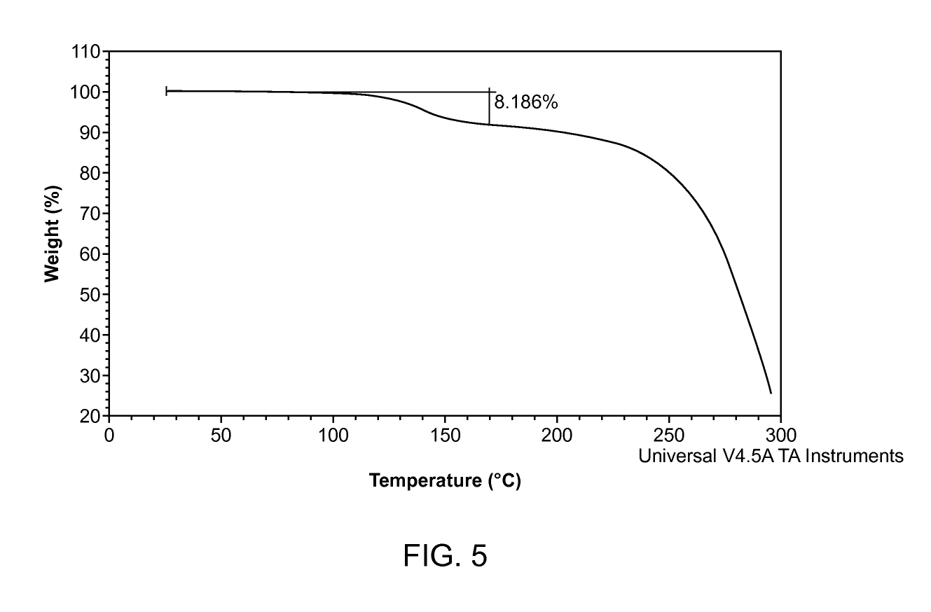

Scale-up of Form II: 60 mg or 40 mg of Compound 1 hydrochloride was dissolved in 3 mL of ethanol or 1-butanol, respectively. Both solutions were filtered, filtrates were collected and then the two filtrates were combined in (1:1) volume ratio. The resulting solution was allowed to evaporate at ambient condition to dryness to yield Form II. Form II was heated to 80° C. by TGA, the XRPD remained unchanged. TGA profile showed a weight loss of 8.19% (FIG. 5) due to HCl loss during heating. DSC profile showed a melting endothermic peak with peak temperature of 189.1° C. and enthalpy of 291.4 J/g (FIG. 6).

Attempted Scale-up of Form III: 60 mg or 40 mg Compound 1 hydrochloride was added in 3 mL of ethanol or acetontrile, respectively. Both solutions were filtered, both filtrates were collected and then combined in (1:1) ratio. The solution was allowed to evaporate at ambient condition to dryness. However, these conditions failed to reproduce Form III as it was formed under similar conditions in initial 96-well plate screening. Presumably, Form III is physically unstable or conversion of Form III to other forms are also possible under the experimental conditions.

### Example 13

### Form II Interconversion Study

Interconversion experiments were performed with the various crystalline forms. Samples of equal amount of Form I mixed with Form II in solvents (ethyl acetate or ethanol) were slurried at ambient condition with covering. The remaining solids were collected by filtration and examined by XRPD. The result showed that some samples turned into identical XRPD pattern as Form I. Under the tested condition, the sole exception was a peak at 18° 2θ. Some observed differences in the —OH stretch region at 3500 cm₋₁ in FTIR were most likely due to the preferred orientation which may be caused by particle size and morphology difference.

While this invention has been described in conjunction with specific embodiments and examples, it will be apparent to a person of ordinary skill in the art, having regard to that skill and this disclosure, that equivalents of the specifically disclosed materials and methods will also be applicable to this invention; and such equivalents are intended to be included within the following claims.

1. A stable crystalline hydrochloride ansolvate of Compound 1:

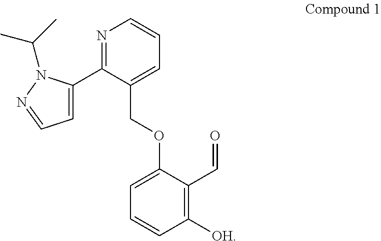 [SMILES: CC(C)n1nccc1-c1ncccc1COc1cccc(O)c1C=O]

2. The stable crystalline ansolvate of Compound 1 of claim 1 that is a crystalline hydrochloride ansolvate.

3. The stable crystalline hydrochloride ansolvate of claim 1, which is substantially free of a solvated polymorph of a hydrochloride of Compound 1.

4. A composition comprising the stable crystalline ansolvate of claim 1.

5. The stable crystalline hydrochloride ansolvate of claim 2 characterized by at least one X-ray powder diffraction peak (Cu Kα radiation) selected from 11.6°, 14.6°, 15.8° and 19.0° 2θ (each ±0.2° 2θ).

6. A method of preparing a stable crystalline hydrochloride ansolvate of claim 1 comprising slurrying a hydrochloride of Compound 1 in methyl etheyl ketone, ethanol or ethyl acetate at room temperature.

7. A method for increasing oxygen affinity of hemoglobin S in a subject, the method comprising administering to a subject in need thereof a therapeutically effective amount of a stable crystalline ansolvate of claim 1.

8. A method for increasing oxygen affinity of hemoglobin S in a subject, the method comprising administering to a subject in need thereof a therapeutically effective amount of a composition of claim 4.

9. A method for treating oxygen deficiency associated with sickle cell anemia, the method comprising administering to a subject in need thereof a therapeutically effective amount of a stable crystalline ansolvate of claim 1.

10. A method for treating oxygen deficiency associated with sickle cell anemia, the method comprising administering to a subject in need thereof a therapeutically effective amount of a composition of claim 4.
